(19) United States
(12) Patent Application Publication
(10) Pub. No.: US 2006/0046978 A1
(43) Pub. Date: Mar. 2, 2006

(54) Novel compounds that inhibit dipeptidyl peptidase (DPP-IV) and neprilysin (NEP) and/or angiotensin converting enzyme (ACE)

(75) Inventors: Sabine Pierau, Freiburg (DE); Christian Oefner, Freiburg (DE); Glenn E. Dale, Basel (CH)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Morphochem AG, Munchen (DE)

(21) Appl. No.: 10/930,606

(22) Filed: Aug. 31, 2004

(51) Int. Cl. A61K 31/69; A61K 31/4439; A61K 31/426; A61K 31/4196; A61K 31/401; A61K 31/165

(52) U.S. Cl. 514/64; 514/343; 514/423; 514/365; 514/383; 514/620

(57) ABSTRACT

This invention relates to novel compounds, compositions containing the compounds, that inhibit dipeptidyl peptidase (especially DPP-IV) and neprilysin (NEP, neutral endopeptidase) as well as dipeptidyl peptidase (especially DPP-IV) and angiotensin converting enzyme (ACE) and/or dipeptidyl Peptidase (especially DPP-IV) and vasopeptidases (especially ACE and NEP). These compounds and pharmaceutical compositions thereof are useful for the treatment as well as the prevention of type 2 diabetes mellitus.

### BACKGROUND OF THE INVENTION

Type 2 insulin-resistant diabetes mellitus accounts for 90-95% of all diabetes. This heterogeneous disorder affects an estimated 6% of the adult population in western society and is expected to grow by 6% per year reaching a total of 200-300 million cases by 2010 (Moller, 2001). A key component of the pathophysiology of type 2 diabetes involves a selective defect in the ability of glucose to induce secretion of insulin from pancreatic β-cells. This defect accounts for the β-cell to compensate for increasing insulin resistance and the development of hyperglycemia. Cells become de-sensitised towards insulin and consequently blood glucose levels stay at abnormally high levels, which in the long term causes severe health problems in these patients. Glucose-dependent insulin secretion is mainly promoted by incretins, predominantly glucose-dependent insulinotropic peptide (GIP) and glucagon-like peptide 1 (GLP-1) (Druker, 2001). These gut peptides are released from the gastrointestinal tract in response to nutrition ingestion and promote nutrient assimilation via potentiation of glucose-dependent insulin secretion (Drucker, 2003). However, insulin secretion has also been shown to be stimulated in a glucose dependent manner by other hormonal peptides including bradykinin (BK) through the kinin-NO pathway as well by atrial natriuretic peptide (ANP) (Arbin 2001, 2003; Yang 1997, Haak 1990; Ruskoaho, 1992).

The available evidence suggests that enhancement of incretin action may be useful for lowering blood glucose in subjects with type 2 diabetes mellitus. Although GLP-1 and GIP have strong potential as chronic therapies for diabetes, both are subject to rapid amino-terminal degradation by dipeptidyl peptidase IV (DPP-IV); In vivo administration of synthetic inhibitors of DPP-IV prevents N-terminal degradation of GLP-1 and GIP, resulting in higher plasma concentrations of these hormones, increased insulin secretion and, therefore, improved glucose tolerance (Marguet et al., 2000; Pospisilik et. al., 2002). BK and ANP are circulating peptides whose activity is regulated due to a large part by neprilysin (NEP). It has been shown that chronic inhibition of NEP by specific (candoxatril) or by vasopeptidase inhibitors (mixanpril and omapatrilat) improves whole body insulin-mediated glucose disposal in insulin-resistant animals. Moreover, like DPP-IV, NEP is involved in the enzymatic inactivation of GLP-1 (Plambaeck et al., 2003; Hupe-Sodmann et al., 1995, 1997). An improvement of GLP-1 stability has been shown by a combined inhibition of NEP and DPP-IV in anaesthetised pigs compared to DPP-IV inhibition alone (Plamboeck et al., 2002). It is proposed that mixed inhibition of NEP and DPP-IV and/or DPP-IV and vaspoeptidases (ACE and NEP) offers an alternative strategy for the treatment of type 2 diabetes.

### SUMMARY OF THE INVENTION

This invention relates to novel compounds with the general formula A-L-B (I), A-L-C (II), A-L-D (III) where
A is an inhibitor of dipeptidyl peptidase or a pharmacophore thereof;
B is an inhibitor of neprilysin or a pharmacophore thereof;
C is an inhibitor of angiotensin converting enzyme (ACE) or a pharmacophore thereof;
D is an inhibitor of vasopeptidases (especially NEP and ACE) or a pharmacophore thereof; and
L is a linker, or a pharmaceutically acceptable salt, solvent, or formulation.

Preferred and/or advantageous embodiments of the invention are described below.

### DETAILED DESCRIPTION

It should be appreciated that certain compounds of Formula (I), (II), or (III) may have tautomeric forms from which only one might be specifically mentioned or depicted in the following description, different geometrical isomers (which are usually denoted as cis/trans isomers or more generally as (E) and (Z) isomers) or different optical isomers as a result of one or more chiral carbon atoms (which are usually nomenclatured under the Cahn-Ingold-Prelog or R/S system). Further, some compounds may display polymorphism. All these tautomeric forms, geometrical or optical isomers (as well as racemates and diastereomers) and polymorphous forms are included in the invention.

The term linker refers to a chemical group which is suitable to couple compound A with either compound B, C, or D. In this case the linker will replace one or more hydrogen atom or chemical groups of the compounds A, B, C, or D. A linker may be a bond, a oxygen or sulfur atom, or can be a group of the chemical formula NH, SO, SO₂, SO₃, SO₄, SO₂NH, PO₄, NHCONH, CONH, CO, COO, NHCOO, an alkylen-, an alkenylen-, an alkinylen-, a heteroalkylen-, a cycloalkylen-, a heterocycloalkylen-, an alkylcycloalkylen-, a heteroalkylcycloalkenylen-, an arylen-, a heteroarylen- or an aralkylen group. The linker can be under physiological conditions metabolically stabile as well as metabolically cleaved.

The term alkyl refers to a saturated or unsaturated (i.e. alkenyl and alkinyl) straight or branched chain alkyl group, containing from one to ten, preferably one to six carbon atoms, for example methyl, ethyl, propyl, iso-propyl, butyl, iso-butyl, sec-butyl, tert-butyl, n-pentyl, iso-pentyl n-hexyl, 2,2-dimethylbutyl, n-octyl, ethenyl (vinyl), propenyl (allyl), iso-propenyl, n-pentyl, butenyl, isoprenyl or hexa-2-enyl; ethynyl, propynyl or butynyl groups. Any alkyl group as defined herein may be substituted with one or more substituents, for example F, Cl, Br, I, NH₂, OH, SH or NO₂.

The term heteroalkyl refers to an alkyl group as defined herein where one or more carbon atoms are replaced by an oxygen, nitrogen, phosphorus or sulphur atom, for example an alkoxy group such as methoxy, ethoxy, propoxy, iso-propoxy, butoxy or tert.-butoxy, an alkoxyalkyl group such as methoxymethyl, ethoxymethyl, 1-methoxyethyl, 1-ethoxyethyl, 2-methoxyethyl or 2-ethoxyethyl, an alkylamino group such as methylamino, ethylamino, propylamino, isopropylamino, dimethylamino or diethylamino, an alkylthio group such as methylthio, ethylthio or isopropylthio or a cyano group. Furthermore, the term heteroalkyl refers to a group derived from a carboxylic acid or carboxylic acid amide such as acetyl, propionyl, acetyloxy, propionyloxy, acetylamino or propionylamino, a carboxyalkyl group such as carboxymethyl, carboxyethyl or carboxypropyl, a carboxyalkyl ester, an alkylthiocarboxyamino group, an alkoxyimino group, an alkylaminothiocarboxyamino group or an alkoxycarbonylamino group. Any heteroalkyl group as defined herein may be substituted with one or more substituents, for example F, Cl, Br, I, NH₂, OH, SH or NO₂.

The term cycloalkyl refers to a saturated or partially unsaturated, cyclic group, having one or more rings, formed by a skeleton that contains from three to 14 carbon atoms, preferably from five or six to ten carbon atoms, for example cyclopropyl, cyclobutyl, cyclopentyl, cyclohexyl, tetralin, cyclopentenyl or cyclohex-2-enyl groups. Any cycloalkyl group as defined herein may be substituted with one or more substituents, for example F, Cl, Br, I, OH, NH₂, SH, N₃, NO₂, alkyl groups such as methyl or ethyl, heteroalkyl groups such as methoxy, methylamino, dimethylamino or cyanide.

The term heterocycloalkyl refers to a cycloalkyl group as defined herein where one or more carbon atoms are replaced by one or more oxygen, nitrogen, phosphorus or sulphur atoms, for example piperidino, morpholino or piperazino groups.

The terms arylalkyl and heteroarylalkyl refer to groups that comprise either aryl or, respectively, heteroaryl as well as alkyl and/or heteroalkyl and/or cycloalkyl and/or heterocycloalkyl groups.

The term aryl refers to an aromatic cyclic group, having one or more rings, formed by a skeleton that contains from five to 14 carbon atoms, preferably from five to ten carbon atoms, for example phenyl or naphthyl groups. Any aryl group as defined herein may be substituted with one or more substituents, for example F, Cl, Br, I, OH, NH₂, SH, N₃, NO₂, alkyl groups such as methyl or ethyl, heteroalkyl groups such as methoxy, methylamino, dimethylamino or cyanide.

The term heteroaryl refers to an aryl group as defined herein where one or more carbon atoms are replaced by an oxygen, nitrogen, boron, phosphorus or sulphur atom, for example pyridyl, imidazolyl, pyrazolyl, quinolinyl, isoquinolinyl, pyrrolyl, oxazolyl, isoxazolyl, thiazolyl, isothiazolyl, 1,2,3-triazolyl, 1,2,4-triazolyl, oxadiazolyl, thiadiazolyl, indolyl, indazolyl, tetrazolyl, pyrazinyl, pyrimidinyl and pyridazinyl groups.

The terms aralkyl or heteroaralkyl refers to groups which contain aryl- or heteroaryl- or alkyl-, alkenyl-, alkinyl- and/or heteroalkyl- and/or cycloalkyl- and/or hetero-cycloalkyl groups as defined herein, for example the tetrahydroisochinolinyl-, benzyl-, 2- or 3-ethyl-indolyl-, 4-methylpyridino-, 2-, 3- or 4-methoxyphenyl-, 2-, 3- or 4-ethoxyphenyl-, 4-carboxyphenylalkyl- or 4-hydroxyphenyl-group.

The expression pharmacophore is known to the expert and for example is described in R. B. Silverman, Medizinische Chemie, VCH Weinheim, 1995, sowie in H.-J. Böhm, G. Klebe, H. Kubinyi, Wirkstoffdesign, Spektrum, Akad. Verl., 1996.

DPP-IV inhibitors are known in the art. For example, DPP-IV inhibitors are generically and specifically disclosed in e.g. in DE19616486 A1, U.S. Pat. No. 6,110,949, U.S. Pat. No. 6,395,767 B2, WO03004496, WO03004498, WO0034241, WO9515309, WO952691, WO03002596, WO03002595, WO03002553, WO03002531, WO03002530, WO03000250, WO03000181, WO03000180, WO02083128, WO02076450, WO02062764, WO02051836, EP1258480, EP1258476, EP1245568, WO0214271, WO0202560, WO0196295, WO0155105, WO0152825, WO0034241, WO0010549, WO9946272, WO9819998, WO9515309, WO0056296, WO0056297, WO0140180, WO0168603, WO0181304, WO0181337, WO0230890, WO0238541, WO02068420, U.S. Pat. No. 6,432,969, WO9308259, WO9310127, WO9116339 WO03033424 A2, WO03033424 A3, WO03040174 A2, and DE10154689 A1. In each case, the subject matter of the final products of the working examples, the pharmaceutical preparations and the claims of the aforementioned patent documents are hereby incorporated into the present application by reference to these publications.

Published patent application WO9819998 discloses N-(N′-substituted glycyl)-2-cyano pyrrolidines, in particular 1-[2-[5-Cyanopyridin-2-yl]amino]-ethylamino]acetyl-2-cyano-(S)-pyrrolidine (NVP-DPP728).

DE19616486 A1 discloses valine-pyrrolidide, valine-thiazolide, isoleucyl-thiazolide, isoleucyl-pyrrolidide, and fumarate salts of isoleucyl-thiazolidide and isoleucyl-pyrrolidide.

Published patent application WO0034241 and published patent U.S. Pat. No. 6,110,949 disclose N-substituted adamantyl-amino-acetyl-2-cyano pyrrolidines and W (substituted glycyl)-4-cyano pyrrolidines respectively. DPP-IV inhibitors of interest are specifically those cited in claims 1 to 4

Published patent application WO9515309 discloses amino acid 2-cyanopyrrolidine amides as inhibitors of DPP-IV. Published patent WO952691 discloses peptidyl derivates of alpha-aminoalkylphosphinic acids, particularly those with proline or related structures.

WO9310127 discloses proline boronic esters useful as DPP-IV inhibitors.

WO03033424 A2, WO03033424 A3, WO03040174 A2, and DE10154689 A1 describe substituted aminoketones as inhibitors of DPP-IV.

Preferred are compounds where A will be taken from the following structural classes: 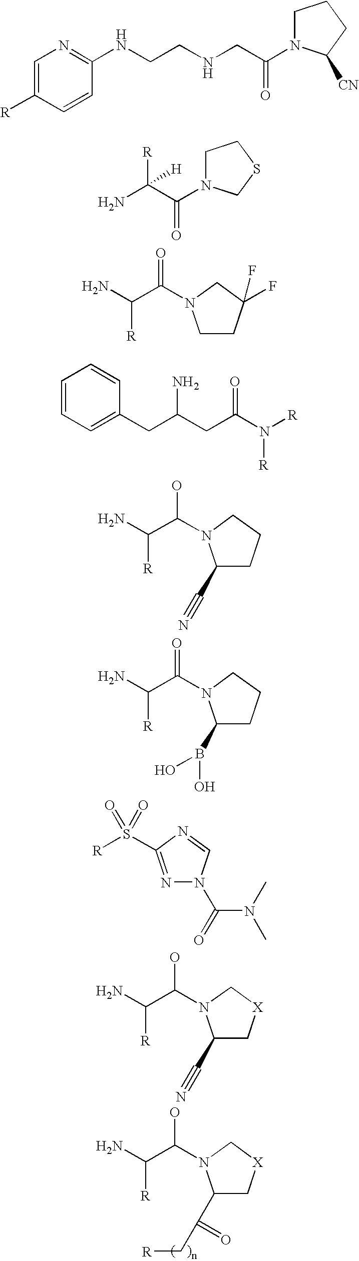
where the remaining groups R are independently chosen as a hydrogen-atom, alkyl-, alkenyl-, alkinyl-, heteroalkyl-, cycloalkyl-, heterocycloalkyl-, alkylcycloalkyl-, heteroalkyl-cycloalkyl-, aryl-, heteroaryl-, aralkyl- or heteroaralkyl groups. Preferentially, L will be linked to R connecting the group B, C, or D to the molecule. X is preferably CH₂, S, SO₂. CHF, CF₂. N is a number between 0 and 5.

The natriuretic peptides constitute a family of peptides that include the atrial (ANP), brain derived (BNP) and C-type natriuretic (CNP) peptides. The natriuretic peptides effect vasodilation, natriuresis, diuresis, decreased aldosterone release, decreased cell growth, and inhibition of the sympathetic nervous system and the renin-angiotensin-aldosterone system indicating their involvement in the regulation of blood pressure and of sodium and water balance. NEP inhibitors impede degradation of natriuretic peptides and elicit pharmacological actions potentially beneficial in the management of cardiovascular disorders. A NEP inhibitor useful in the said combination is selected from a group of compounds represented by candoxatril, sinorphan, thiorphan, SCH34826 and SCH42495. NEP inhibitors are known in the art. For example, NEP Inhibitors are generically and specifically disclosed in e.g. WO02079143, WO0144225 (Omapatrilat), WO9738705, WO9724342, WO9724341, WO9719102, WO9700261, WO9622998, WO9535315, WO9535307, WO9535302, WO9501353, WO9417036, WO02092622, WO0160822, WO9955726, JP200256318, U.S. Pat. No. 5,952,327, U.S. Pat. No. 5,939,446, WO9919320, U.S. Pat. No. 5,760,241, EP636630, U.S. Pat. No. 5,438,046 EP0738711, JP96277271, JP96245609, JP96231516, JP96225586, EP733642, JP96208646, EP728746, EP723974, U.S. Pat. No. 5,523,313, WO9614293, JP96041015, WO03002595, WO03002596, WO03004498, WO03000181, WO03000180, WO02083128, WO02076450, WO0168603, WO0140180, WO9515309, WO9308259. In each case, the subject matter of the final products of the working examples, the pharmaceutical preparations and the claims of the aforementioned patent documents are hereby incorporated into the present application by reference to these publications.

Preferred are compounds where B will be taken from the following structural classes: 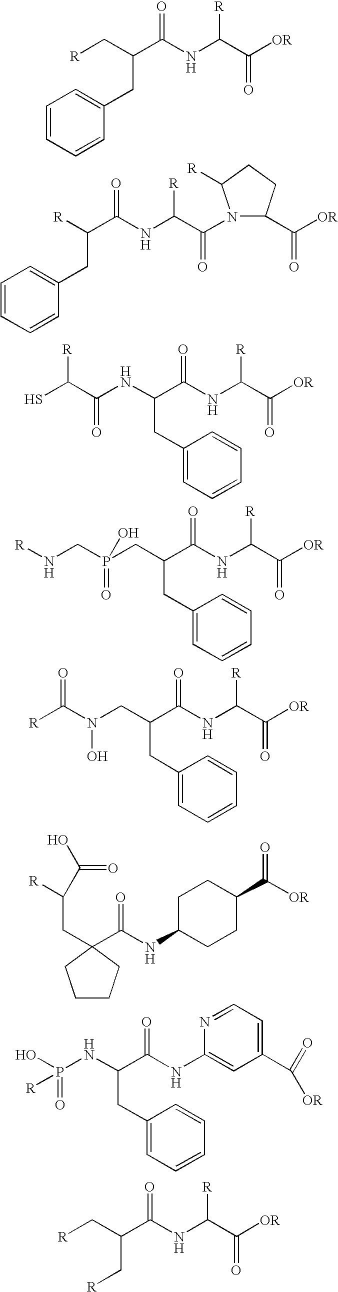
where by the group R is independently chosen from hydrogen atom, alkyl-, alkenyl-, alkinyl-, heteroalkyl-, cycloalkyl-, heterocycloalkyl-, alkylcycloalkyl-, heteroalkylcycloalkyl-, aryl-, heteroaryl-, aralkyl-oder heteroaralkyl group. Preferentially, the linker L will be connected through R to group A.

The interruption of the enzymatic degradation of angiotensin I to angiotensin II with so called ACE-inhibitors (also called angiotensin converting enzyme inhibitors) is a successful variant for the regulation of blood pressure and also a therapeutic method for treatment of congestive heart failure.

The classes of ACE inhibitors are known in the art. For example, mention may be made of the compounds selected from the group consisting of alacepril, benazepril, benazeprilat, captopril, cerenopril, cilazapril, delapril, enalapril, enalaprilat, fosinopril, fosinoprilat, imidapril, lisinopril, moexipril, moveltopril, pentopril, perindopril, quinapril, quinaprilat, ramipril, ramiprilat, spirapril, temocapril, trandolapril, and zofenopril, or, in each case, a pharmaceutically acceptable salt thereof.

Preferred are compounds where C will be taken from the following structural classes: 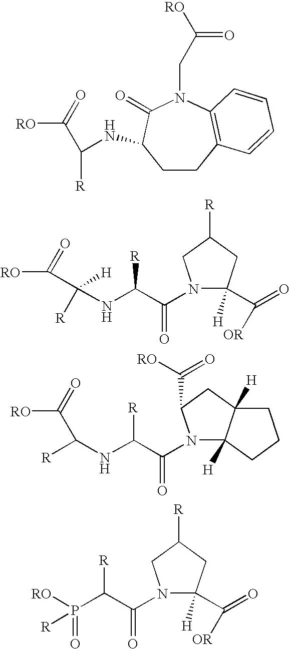
where by the group R is independently chosen from hydrogen atom, alkyl-, alkenyl-, alkinyl-, heteroalkyl-, cycloalkyl-, heterocycloalkyl-, alkylcycloalkyl-, heteroalkylcycloalkyl-, aryl-, heteroaryl-, aralkyl-oder heteroaralkyl group. Preferentially, the linker L is connected through R to group A.

Compounds having inhibitory effects on both angiotensin converting enzyme and neutral endopeptidase, so called dual ACE/NEP inhibitors (or vasopeptidase inhibitors), can be used for the treatment of cardiovascular pathologies. A preferred dual angiotensin converting enzyme/neutral endopeptidase inhibitor is, for example, omapatrilat, sampatrilat, fasidotril or fasidotrilat, SQ28133, M-100240, Z-13752A, BMS-189921, ER-40121, CGS-30440, SA-7060, compounds described in NEP patents and following patents JP-96231516, JP-96225586, JP-96277271, CGS-28106.

Preferred are compounds where D will be taken from the following structural classes: 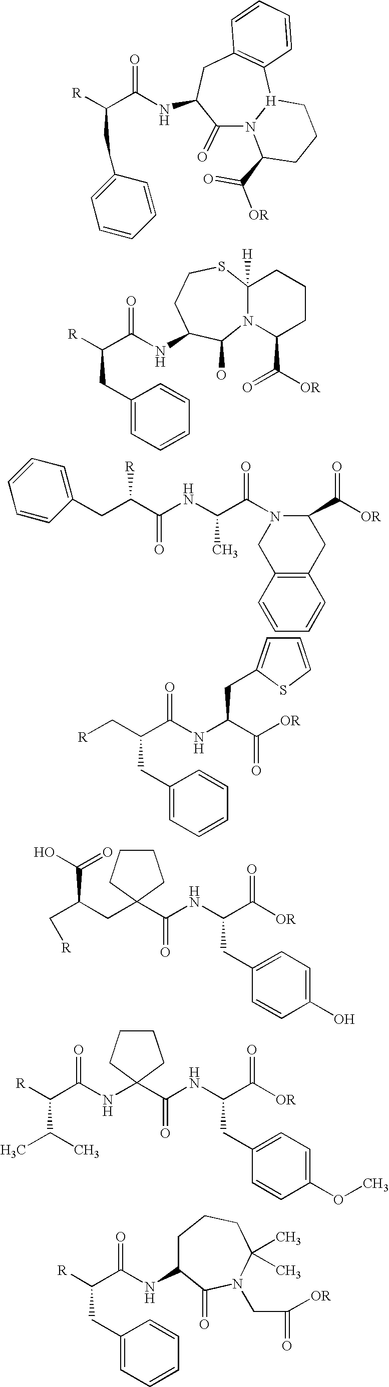 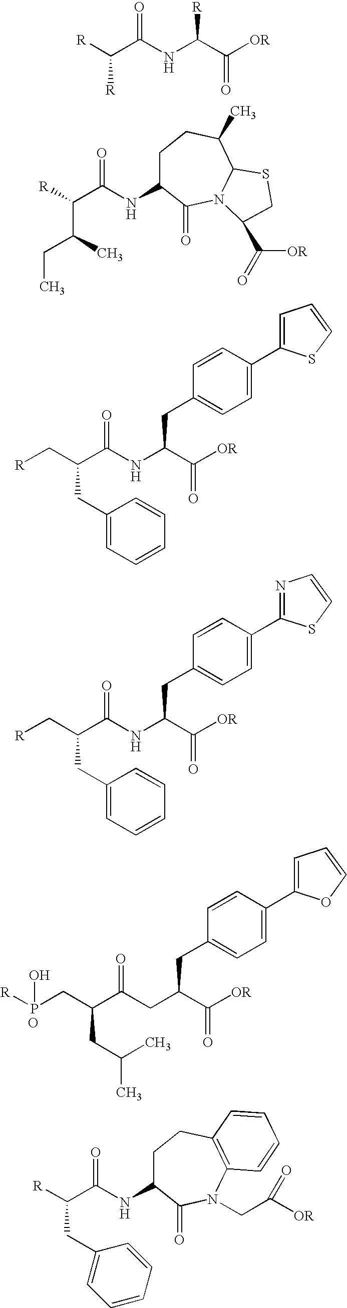 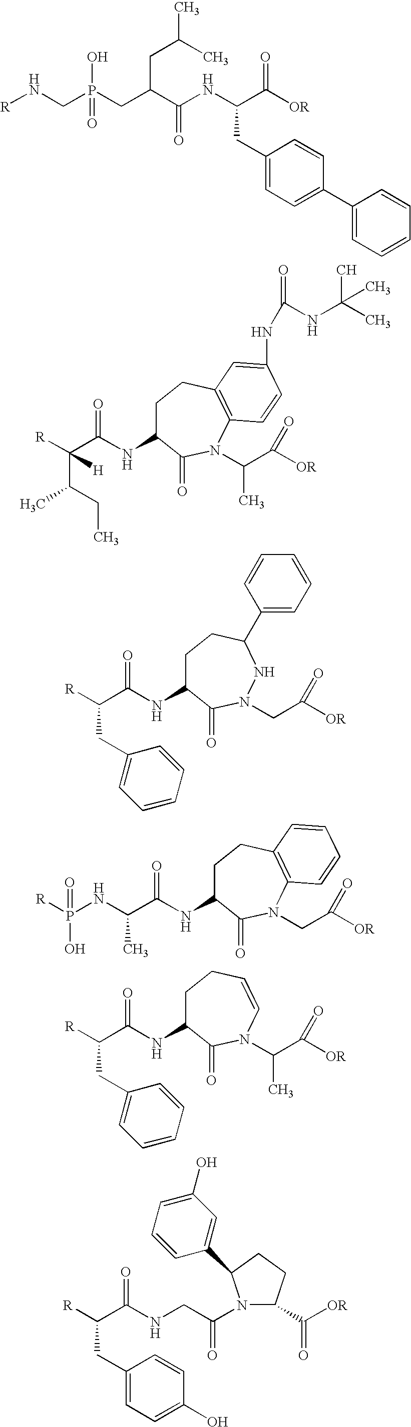 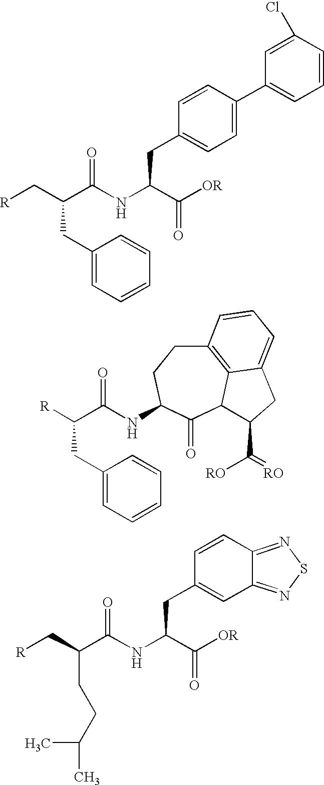
where by the group R is independently chosen from hydrogen atom, alkyl-, alkenyl-, alkinyl-, heteroalkyl-, cycloalkyl-, heterocycloalkyl-, alkylcycloalkyl-, heteroalkylcycloalkyl-, aryl-, heteroaryl-, aralkyl-oder heteroaralkyl group. Preferentially, the linker L is connected through R to group A.

The present invention also relates to pharmacologically acceptable salts, or solvates and hydrates, respectively, and to compositions and formulations of compounds of Formula (I), (II), or (III). The present invention describes procedures to produce pharmaceutically useful agents, which contain these compounds, as well as the use of these compounds for the production of pharmaceutically useful agents.

The pharmaceutical compositions according to the present invention contain at least one compound of Formula (I), (II), or (III) as the active agent and optionally carriers and/or diluents and/or adjuvants.

Examples of such pharmacologically acceptable salts of sufficiently basic compounds of Formula (I), (II), or (III) are salts of physiologically acceptable mineral acids like hydrochloric, hydrobromic, sulfuric and phosphoric acid; or salts of organic acids like methanesulfonic, p-toluenesulfonic, lactic, acetic, trifluoroacetic, citric, succinic, fumaric, maleic and salicylic acid. Further, a sufficiently acid compound of Formula (I), (II), or (III) may form alkali or earth alkaline metal salts, for example sodium, potassium, lithium, calcium or magnesium salts; ammonium salts; or organic base salts, for example methylamine, dimethylamine, trimethylamine, triethylamine, ethylenediamine, ethanolamine, choline hydroxide, meglumin, piperidine, morpholine, tris-(2-hydroxyethyl)amine, lysine or arginine salts. Compounds of Formula (I), (II), or (III) may be solvated, especially hydrated. The hydratisation can occur during the process of production or as a consequence of the hygroscopic nature of the initially water free compounds of Formula (I), (II), or (III). The compounds of Formula (I), (II), or (III) contain asymmetric C-atoms and may be present either as achiral compounds, mixtures of diastereomers, mixtures of enantiomers or as optically pure compounds.

The present invention also relates to pro-drugs which are composed of a compound of Formula (I), (II), or (III) and at least one pharmacologically acceptable protective group which will be cleaved off under physiological conditions, such as an alkoxy-, aralkyloxy-, acyl-, acyloxymethyl group (e.g. pivaloyloxymethyl), an 2-alkyl-, 2-aryl- or 2-aralkyl-oxycarbonyl-2-alkylidene ethyl group or an acyloxy group as defined herein, e.g. ethoxy, benzyloxy, acetyl or acetyloxy.

The compounds of Formula (I), (II), or (III) and pharmaceutical compositions of this invention may have utility in the treatment of Type 2 diabetes and in the treatment and prevention of the numerous conditions that often accompany Type 2 diabetes, including metabolic syndrome X, reactive hypoglycemia, diabetic dislipidemia, and obesity.

The compounds of Formula (I), (II), or (III) and pharmaceutical compositions of this invention may have utility in the treatment, controlling or prevention of one or more conditions selected from the group consisting of: (1) hyperglycemia, (2) low glucose tolerance, (3) insulin resistance, (4) obesity, (5) lipid disorders, (6) dyslipidemia, (7) hyperlipidemia, (8) hypertriglyceridemia, (10) low HDL levels, (11) high HDL levels, (12) atherosclerosis and its sequelae, (13) vascular restenosis, (14) irritable bowel syndrome, (15) inflammatory bowel disease, including Crohn's disease and ulcerative colitis, (16) other inflammatory conditions, (17) pancreatitis, (18) abdominal obesity, (19) neurodegenerative disease, (20) retinopathy, (21) nephropathy, (22) neuropathy, (23) syndrome X, (24) ovarian hyperandrogenism (polycystic ovarian syndrome), (25) Type 2 diabetes, (26) growth hormone deficiency, (27) neutropenia, (28) neuronal disorders, (29) tumor metastasis, (30) benign prostatic hypertrophy, (31) gingivitis, (32) hypertension, (33) osteoporosis, and other disorders where insulin resistance is a component or that may be treated by inhibitors of DPP-IV, NEP, and/or ACE.

As mentioned above, therapeutically useful agents that contain compounds of Formula (I), (II), or (III), their solvates, salts and formulations are also comprised in the scope of the present invention. In general, compounds of Formula (I), (II), or (III) will be administered by using the known and acceptable modes known in the art, either alone or in combination with any other therapeutic agent. Such therapeutically useful agents can be administered by one of the following routes: oral, e.g. as tablets, dragees, coated tablets, pills, semisolids, soft or hard capsules, for example soft and hard gelatine capsules, aqueous or oily solutions, emulsions, suspensions or syrups, parenteral including intravenous, intramuscular and subcutaneous injection, e.g. as an injectable solution or suspension, rectal as suppositories, by inhalation or insufflation, e.g. as a powder formulation, as microcrystals or as a spray (e.g. liquid aerosol), transdermal, for example via an transdermal delivery system (TDS) such as a plaster containing the active ingredient or intranasal. For the production of such tablets, pills, semisolids, coated tablets, dragees and hard, e.g. gelatine, capsules the therapeutically useful product may be mixed with pharmaceutically inert, inorganic or organic excipients as are e.g. lactose, sucrose, glucose, gelatin, malt, silica gel, starch or derivatives thereof, talc, stearinic acid or their salts, dried skim milk, and the like. For the production of soft capsules one may use excipients as are e.g. vegetable, petroleum, animal or synthetic oils, wax, fat, polyols. For the production of liquid solutions, emulsions or suspensions or syrups one may use excipients as are e.g. water, alcohols, aqueous saline, aqueous dextrose, polyols, glycerin, cyclodextrins, vegetable, petroleum, animal or synthetic oils. For suppositories one may use excipients as are e.g. vegetable, petroleum, animal or synthetic oils, wax, fat and polyols. For aerosol formulations one may use compressed gases suitable for this purpose, as are e.g. oxygen, nitrogen and carbon dioxide. The pharmaceutically useful agents may also contain additives for conservation, stabilisation, e.g. UV stabilizers, emulsifiers, sweeteners, aromatisers, salts to change the osmotic pressure, buffers, coating additives and antioxidants.

In the following the invention is described in more detail with reference to examples. These examples are intended for illustration only and are not to be construed as any limitation.

### EXAMPLES

### Example 1

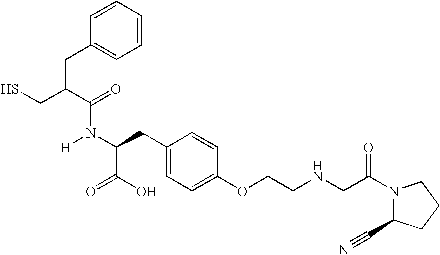

### (2-Hydroxyethylamino)-acetic acid

Chloro acetic acid (11 g, 116 mmol) was added dropwise to a solution of ethanolamine (17.6 mL, 292 mmol) in water (14 mL). After stirring for 24 h at rt, the solution was evaporated and the product crystallised with ethanol. The crude product was recrystallised from ethanol/water.

¹H-NMR (300 MHz, D₂O) 3.76 (dd, J=5.25 Hz, 2H); 3.56 (s, 2H); 3.12 (dd, J=5.25 Hz, 2H); MS—(M−H⁺) 120.3.

### [tert-Butoxycarbonyl-(2-hydroxyethyl)-amino]-acetic acid

Di-tert-butyl-dicarbonate (6.83 g, 31.3 mmol) was added to a solution of (2-hydroxyethylamino)acetic acid (3.39 g, 28.4 mmol) in 1 N NaOH (28.4 mL, 28.4 mmol), water (28.4 mL) and dioxane (56.8 mL). After stirring for 24 h at rt, the solution was partially evaporated, acidified with 1 N HCl (pH 1) and extracted several times with ethyl acetate. The combined organic layers were dried over MgSO₄, filtered and evaporated.

¹H-NMR (300 MHz, CDCl₃) 1.42, 1.45 (d, 9H,); 3.45, 3.64 (m, 2H,); 3.77, 4.41(m, 2H,); 3.95, 4.00(d, 2H,); 7.17 (b, 2H,); MS—(MH+) 220.3, (M−H⁻) 218.5.

### {tert-Butoxycarbonyl-[2-(tetrahydropyran-2-yloxy)-ethyl]-amino}-acetic acid

3,4-Dihydro-2H-pyrane (3.1 mL, 34.2 mmol) and Amberlyst H-15 (0.8 g, 0.01 eq) were added to a solution of [tert-Butoxycarbonyl-(2-hydroxyethyl)-amino]-acetic acid (6.08 g, 27.7 mmol) in dichloromethane (14 mL). After stirring for 24 h at rt, the solution was filtered and evaporated.

MS—(M−H⁺) 304.1; (M−H⁻) 302.5.

### [2-(2-Carbamoylpyrrolidin-1-yl)-2-oxo-ethyl]-[2-(tetra-hydropyran-2-yloxy)-ethyl]-carbamic acid tert.-butylester

{tert-Butoxycarbonyl-[2-(tetrahydropyran-2-yloxy)-ethyl]-amino}-acetic acid (2.404 g, 7.92 mmol) and L-prolinamide (1 g, 8.76 mmol) were suspended in dichloromethane. Then 1-(3-Dimethylaminopropyl)-3-ethylcarbodiimide hydrochloride (1.826 g, 9.52 mmol), 1-hydroxybenzotriazole (1.612 g, 11.9 mmol) and N-methylmorpholine (2.6 mL, 23.6 mmol) were added. After stirring for 24 h at rt, the solution was concentrated and purified by column chromatography (silica gel, dichloromethane/ethanol/ammonium hydroxide).

MS—(M−H⁺) 400.4.

### [2-(2-Cyanopyrrolindin-1-yl)-2-oxoethyl]-[2-(tetrahydro-pyran-2-yloxy)-ethyl]-carbamic acid tert.-butylester

[2-(2-Carbamoylpyrrolindin-1-yl)-2-oxoethyl]-[2-(tetrahydropyran-2-yloxy)ethyl] carbamic acid tert.-butylester (400 mg, 1 mmol) and imidazole (72 mg, 1.3 mmol) were dissolved in pyridine (8 mL) and cooled to −5° C. Then phosphorus oxychloride (0.12 mL, 1.1 mmol) was added and the solution was warmed to rt. After 90 min the solution was concentrated, the residue dissolved in ethyl acetate and washed with 5% aqueous citric acid. The aqueous layer was extracted several times with ethyl acetate and the combined organic layers were dried over MgSO₄, filtered and evaporated.

MS—(M−H⁺) 382.6.

### [2-(2-Cyanopyrrolindin-1-yl)-2-oxoethyl]-(2-hydroxyethyl)-carbamic acid tert.-butylester

To a solution of [2-(2-Cyanopyrrolindin-1-yl)-2-oxo-ethyl]-[2-(tetrahydropyran-2-yloxy)-ethyl]-carbamic acid tert.-butylester (392 mg, 1 mmol) in ethanol (11 mL) was added pyridinium para-toluenesulfonate (26 mg, 0.1 mmol) and warmed to 60° C. After 6 h the solution was cooled to rt and filtered over a short silica gel column (dichloromethane/methanol).

MS—(M−H⁺) 298.6.

### 2-Benzyl-2-hydroxymethyl-diethyl-malonate

Paraformaldehyde (1.306 g, 43.5 mmol) and potassium hydroxide (8 mg, 0.14 mmol) were added to diethylbenzylmalonate (9.3 mL, 40.0 mmol) and warmed to 80° C. After 24 h the solution was cooled to rt, diluted with chloroform and filtered over a short silica gel column.

MS—(M−H⁺) 281.4.

### 2-Benzylacrylic acid

2-Benzyl-2-hydroxymethyl-diethyl-malonate (11.2 g, 40.0 mmol) was dissolved in a mixture of 48% HBr (23 mL, 420 mmol) and acetic acid (7.6 mL, 130 mmol) and warmed to 80° C. After 3 days the solution was cooled to rt and diluted with dichloromethane. The organic layer was washed with water and brine, dried over MgSO₄, filtered and evaporated. The crude product was purified by column chromatography (silica gel, hexanes/ethyl acetate).

MS—(M−H⁻) 161.4.

### 2-Acetylsulfanylmethyl-3-phenylpropionic acid

2-Benzylacrylic acid (2.55 g, 15.8 mmol) was dissolved in thioacetic acid (2.81 mL, 39.4 mmol). The solution was stirred for 90 min at rt, for 1 h at 50° C. and then concentrated. The crude product was purified by column chromatography (silica gel, hexanes/ethyl acetate).

### 2-(2-Acetylsulfanylmethyl-3-phenylpropionylamino)-3-(4-hydroxyphenyl)-propionic acid methy lester

2-Acetylsulfanylmethyl-3-phenylpropionic acid (0.5 g, 2.1 mmol) and L-tyrosine methy lester (0.87 g, 4.46 mmol) were suspended in dimethylformamide (5 mL). Then HBTU (0.8 g, 2.1 mmol) and triethylamine (0.59 g, 4.2 mmol) were added. After stirring for 4 h at rt, the reaction mixture was diluted with ethyl acetate and washed with 1 N HCl, sat. bicarbonate and brine, dried over MgSO₄, filtered and evaporated. The crude product was purified by column chromatography (silica gel, hexanes/ethyl acetate).

MS—(M−H⁺) 416.9; (M−H⁻) 414.5.

### 2-(3-Acetylsulfanyl-2-benzylpropionylamino)-3-[4-(2-{tert-butoxycarbonyl-[2-(2-cyanopyrrolidin-1-yl)-2-oxoethyl]-amino}-ethoxy)-phenyl]-propionic acid methyl ester

To a solution of [2-(2-Cyanopyrrolidin-1-yl)-2-oxoethyl]-(2-hydroxyethyl)-carbamic acid tert.-butyl ester (100 mg, 0.34 mmol) in THF (2.1 mL) was added triphenyl-phosphine (85.5 mg, 0.33 mmol) and diethyl azodicarboxylate (0.04 mL, 0.33 mmol). After stirring for 1 h at rt, 2-(2-Acetylsulfanylmethyl-3-phenylpropionylamino)-3-(4-hydroxyphenyl)-propionic acid methyl ester (104 mg, 0.25 mmol) was added. After 2 days more triphenylphosphine (131 mg, 0.50 mmol) and diethyl azodicarboxylate (0.06 mL, 0.50 mmol) were added. After another 24 h the solution was partially evaporated and the residue triturated with ether. The solution was filtered and evaporated. The crude product was purified by column chromatography (silica gel, hexanes/ethyl acetate, dichloro-methane/methanol).

MS—(M−H⁺) 695.3.

### 2-(3-Acetylsulfanyl-2-benzylpropionylamino)-3-(4-{2-[2-(2-cyanopyrrolidin-1-yl)-2-oxoethylamino]-ethoxy}-phenyl)-propionic acid methyl ester

2-(3-Acetylsulfanyl-2-benzylpropionylamino)-3-[4-(2-{tert-butoxycarbonyl-[2-(2-cyanopyrrolidin-1-yl)-2-oxoethyl]-amino}-ethoxy)-phenyl]-propionic acid methylester (46 mg, 0.066 mmol) was dissolved in trifluoroacetic acid (1 mL, 13 mmol) and water (0.05 mL). After stirring for 3 h at rt, the solution was evaporated and the crude product was purified by column chromatography (silica gel, dichloromethane/ethanol/ammonium-hydroxide).

MS—(M−H⁺) 595.7.

### 2-(2-Benzyl-3-mercaptopropionylamino)-3-(4-{2-[2-(2-cyanopyrrolidin-1-yl)-2-oxoethylamino]-ethoxy}-phenyl)-propionic acid

N lithium hydroxide (0.454 mL, 0.0454 mmol) was added to a solution of 2-(3-Acetylsulfanyl-2-benzylpropionylamino)-3-(4-{2-[2-(2-cyanopyrrolidin-1-yl)-2-oxoethyl-amino]-ethoxy}-phenyl)-propionic acid methyl ester (9 mg, 0.015 mmol) in tetrahydrofuran (0.13 mL). After stirring for 24 h at rt, the solution was concentrated in vacuo.

MS—(M−H⁺) 540.8; (M−H⁻) 538.8.

### Example 2

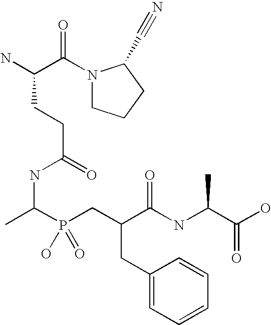

### 2-tert-Butoxycarbonylamino-glutaric acid-5-ethyl ester

L-Glutamic acid-5-ethylester (5.4 g, 30.8 mmol) and di-tert-butyl-dicarbonate (8.1 g, 37 mmol) were suspended in methanol (42 ml) and triethylamine (5 ml) and warmed to 45° C. until everything is dissolved. The solution was then stirred for 30 min at rt and concentrated in vacuo. The residue was dissolved in water, acidified with 10% aq. sodium hydrogen sulfate solution and extracted several times with ethyl acetate. The combined organic layers were dried over MgSO₄, filtered and evaporated.

¹H-NMR (300 MHz, CDCl₃) 9.56 (bs, 1H), 5.25 (bd, 1H), 4.35-4.34 (m, 1H), 4.13 (q, 2H), 2.49-2.38 (m, 2H), 2.26-2.2 (m, 1H), 2.05-1.98 (m, 1H), 1.45 (s, 9H), 1.26 (t, 3H)

MS—(M−H⁺) 276.5; (M−H⁻) 274.5

### 4-tert-Butoxycarbonylamino-5-(2-carbamoylpyrrolidin-1-yl)-5-oxopentanoic acid ethyl ester

2-tert-Butoxycarbonylamino-glutaric acid-5-ethyl ester (4.2 g, 15.2 mmol) was dissolved in dichloromethane (50 ml) and cooled to −20° C. Then N-methylmorpholine (3.4 ml, 30.4 mmol) and isobutyl chloroformate (2 ml, 15.2 mmol) were added and the solution stirred for 30 min at −20° C. After the addition of L-prolinamide (1.74 g, 15.2 mmol), the reaction mixture was stirred for 1 h at −20° C., then over night at rt. After evaporation the crude product was purified by column chromatography (silica gel, dichloromethane/methanol).

MS—(M−H⁺) 372.4

### 4-tert-Butoxycarbonylamino-5-(2-cyanopyrrolidin-1-yl)-5-oxopentanoic acid ethyl ester

4-tert-Butoxycarbonylamino-5-(2-carbamoylpyrrolidin-1-yl)-5-oxopentanoic acid ethyl ester (3.8 g, 10.2 mmol) was dissolved in pyridine (75 ml) and cooled to −20° C. Then imidazole (0.73 g, 10.7 mmol) and phosphorus oxychloride (1.26 ml, 13.8 mmol) were added and the reaction mixture warmed to rt. After 4 h the reaction mixture was concentrated and the crude product was purified by column chromatography (silica gel, hexanes/ethyl acetate).

MS—(M−H⁺) 354.6

### 4-tert-Butoxycarbonylamino-5-(2-cyanopyrrolidin-1-yl)-5-oxopentanoic acid

4-tert-Butoxycarbonylamino-5-(2-cyanopyrrolidin-1-yl)-5-oxopentanoic acid ethyl ester (3.3 g, 9.37 mmol) was dissolved in THF (50 ml) and 0.1 N LiOH (94 ml) and stirred for 5 h at rt. THF was evaporated and the aqueous layer acidified with 10% aq. sodium hydrogen sulfate solution and extracted several times with ethyl acetate The combined organic layers were dried over MgSO₄, filtered and evaporated.

MS—(M−H⁺) 326.8; (M−H⁻) 324.9

### (1-tert-Butoxycarbonylaminoethyl)-phosphinic acid

(1-Aminoethyl)-phosphinic acid (synthesized according literature: Dingwall et al, J. Chem. Soc. Perkin Trans. I, 1984, 2845) (9.3 g, 85 mmol) was suspended in dioxane (170 ml) and water (85 ml), then 1 N NaOH (85 ml) and di-tert-butyl-dicarbonate (22.3 g, 102 mmol) were added and the solution stirred at rt over night. The solution was partially evaporated, acidified to pH 1 with 2 M HCl, saturated with NaCl and extracted serveral times with ethyl acetate. The combined organic layers were dried over MgSO₄, filtered and evaporated.

¹H-NMR (300 MHz, DMSO-d₆) 7.14 (bd, 1H), 3.59-3.57 (m, 1H), 1.38 (s, 9H), 1.91-1.11 (m, 3H)

MS—(M−H⁺) 210.3; (M−H⁻) 207.9

### 2-Benzylacrylic acid ethyl ester

To a solution of diethyl benzylmalonate (23.3 ml, 100 mmol) in ethanol (64 ml), was added dropwise a solution of KOH (5.6 g, 100 mmol) in ethanol (64 ml) and the resulting mixture was stirred for 12 h at rt. The solution was partially evaporated and the residue dissolved in water (10 ml). Conc. HCl was added at 0° C. and the solution extracted several times with ether. The combined organic layers were dried over MgSO₄, filtered and evaporated. Pyridine (18 ml), piperidine (1 ml, 10 mmol) and paraformaldehyde (2.5 g, 83 mmol) were added to the residue and heated under reflux until the gas evolution ceased. After cooling to rt the reaction mixture was diluted with water (300 ml) and extracted several times with pentane. The combined organic layers were washed with 1 N HCl, water, sat. bicarbonate, water and brine, dried over MgSO₄, filtered and evaporated. The crude product was purified by column chromatography (silica gel, hexanes/ethyl acetate).

¹H-NMR (300 MHz, CDCl₃) 7.34-7.21 (m, 5H), 6.25 (d, 1H), 5.47 (d, 1H), 4.17 (q, 2H), 3.66 (s, 2H), 1.28 (t, 3H)

### 2-Benzyl-3-[(1-tert-butoxycarbonylaminoethyl)-hydroxyphosphinoyl]-propionic acid ethyl ester

2-Benzylacrylic acid ethyl ester (18.6 g, 98 mmol) and (1-tert-Butoxycarbonylaminoethyl)-phosphinic acid (17.1 g, 82 mmol) were dissolved in N,O-bistrimethylsilyl acetamide (50 ml) and stirred over night at rt (tlc control). The reaction mixture was diluted with water and extracted several times with ethyl acetate. The combined organic layers were washed with brine, dried over MgSO₄, filtered and evaporated. The crude product was purified by column chromatography (silica gel, hexanes/ethyl acetate).

MS—(M−H⁺) 400.5; (M−H⁻) 398.1

### 2-Benzyl-3-[(1-tert-butoxycarbonylamino-ethyl)-hydroxy-phosphinoyl]-propionic acid

2-Benzyl-3-[(1-tert-butoxycarbonylamino-ethyl)-hydroxy-phosphinoyl]-propionic acid ethyl ester (28 g, 70 mmol) was dissolved in ethanol (350 ml) and 1 N NaOH (350 ml) and stirred at rt over night. The solution was partially evaporated, acidified with 10% aq. sodium hydrogen sulfate solution, saturated with NaCl and extracted several times with ethyl acetate. The combined organic layers were dried over MgSO₄, filtered and evaporated.

MS—(M−H⁺) 372.3; (M−H⁻) 370.4

### 2-{2-Benzyl-3-[(1-tert-butoxycarbonylaminoethyl)-hydroxyphosphinoyl]-propionylamino}-propionic acid methyl ester

2-Benzyl-3-[(1-tert-butoxycarbonylaminoethyl)-hydroxyphosphinoyl]-propionic acid (3 g, 8.1 mmol) and L-alanine methyl ester (1.4 g, 9.7 mmol) were dissolved in dimethylformamide (60 ml). Then benzotriazol-1-yloxy-tris(dimethylamino)-phosphonium hexafluorophosphate (4.5 g, 10.1 mmol) and triethylamine (2.7 ml, 20.3 mmol) were added and the reaction mixture stirred at rt (reaction control with HPLC-MS). The reaction mixture was diluted with ethyl acetate and washed with 1 N HCl and brine. The combined aqueous layers were saturated with NaCl and extracted several times with ethyl acetate. The combined organic layers were dried over MgSO₄, filtered and evaporated. The crude product was purified by column chromatography (silica gel, ethyl acetate, dichloromethane/methanol, methanol).

MS—(M−H⁺) 457.9; (M−H⁻) 455.5

### 2-{2-Benzyl-3-[(1-tert-butoxycarbonylaminoethyl)-hydroxyphosphinoyl]-propionylamino}-propionic acid hydrochloride

2-{2-Benzyl-3-[(1-tert-butoxycarbonylaminoethyl)-hydroxyphosphinoyl]-propionylamino}-propionic acid methyl ester (2.66 g, 5.8 mmol) was dissolved in methanolic HCl (1.25 M, 14 ml) and stirred at rt over night. The solution was evaporated and the product dried under high vacuum.

MS—(M−H⁺) 357.4; (M−H⁻) 355.5

### 2-[2-Benzyl-3-({1-[4-tert-butoxycarbonylamino-5-(2-cyanopyrrolidin-1-yl)-5-oxopentanoylamino]-ethyl}-hydroxyphosphinoyl)-propionylamino]-propionic acid methyl ester

2-{2-Benzyl-3-[(1-tert-butoxycarbonylaminoethyl)-hydroxyphosphinoyl]-propionylamino}-propionic acid hydrochloride (1 g, 2.55 mmol) and 4-tert-Butoxycarbonylamino-5-(2-cyanopyrrolidin-1-yl)-5-oxopentanoic acid (992 mg, 3.05 mmol) were dissolved in dichloromethane (4 ml). Then N-methylmorpholine (0.84 ml, 7.64 mmol), 1-(3-Dimethylaminopropyl)-3-ethylcarbodiimide hydrochloride (635 mg, 3.31 mmol) and hydroxybenzotriazole (413 mg, 3.05 mmol) were added and the reaction mixture stirred at rt over night. The reaction mixture was diluted with ethyl acetate and washed with 1 N HCl and brine. The combined aqueous layers were saturated with NaCl and extracted several times with ethyl acetate. The combined organic layers were dried over MgSO₄, filtered and evaporated. The crude product was purified by column chromatography (silica gel, ethyl acetate, dichloromethane/methanol, methanol).

MS—(M−H⁺) 664.5; (M−H⁻) 662.7

### 2-[2-Benzyl-3-({1-[4-tert-butoxycarbonylamino-5-(2-cyanopyrrolidin-1-yl)-5-oxopentanoylamino]-ethyl}-hydroxyphosphinoyl)-propionylamino]-propionic acid

2-[2-Benzyl-3-({1-[4-tert-butoxycarbonylamino-5-(2-cyanopyrrolidin-1-yl)-5-oxopentanoylamino]-ethyl}-hydroxyphosphinoyl)-propionylamino]-propionic acid methyl ester (200 mg, 0.3 mmol) was dissolved in ethanol (1.5 ml) and 1 N NaOH (1.5 ml) and stirred at rt (careful reaction control with HPLC-MS). The solution was partially evaporated, acidified with 10% aq. sodium hydrogen sulfate solution, saturated with NaCl and extracted serveral times with ethyl acetate. The combined organic layers were dried over MgSO₄, filtered and evaporated.

MS—(M−H⁺) 651.0

### 2-[3-({1-[4-Amino-5-(2-cyanopyrrolidin-1-yl)-5-oxopentanoylamino]-ethyl}-hydroxy-phosphinoyl)-2-benzylpropionylamino]-propionic acid trifluoroacetate

2-[2-Benzyl-3-({1-[4-tert-butoxycarbonylamino-5-(2-cyanopyrrolidin-1-yl)-5-oxopentanoylamino]-ethyl}-hydroxyphosphinoyl)-propionylamino]-propionic acid (160 mg, 0.25 mmol) was dissolved in trifluoro acetic acid (1 ml) and stirred a rt (reaction control with HPLC-MS). After the reaction was complete, the solution was evaporated and the product dried under high vacuum.

MS—(M−H⁺) 551.0

### Example 3

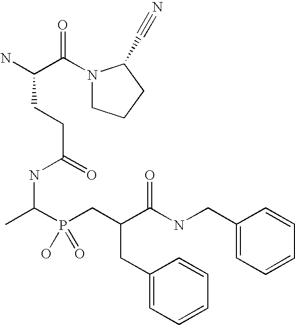

### (2-Benzylcarbamoyl-3-phenylpropyl)-(1-tert-butoxycarbonylaminoethyl)-phosphinic acid

2-Benzyl-3-[(1-tert-butoxycarbonylaminoethyl)-hydroxyphosphinoyl]-propionic acid (1 g, 2.7 mmol) and benzylamine (0.35 ml, 3.2 mmol) were dissolved in dimethylformamide (20 ml). Then benzotriazol-1-yloxy-tris(dimethylamino)-phosphonium hexafluorophosphate (1.5 g, 3.4 mmol) and triethylamine (0.9 ml, 6.8 mmol) were added and the reaction mixture stirred at rt (reaction control with HPLC-MS). The reaction mixture was diluted with ethyl acetate and washed with 1 N HCl and brine. The combined aqueous layers were saturated with NaCl and extracted several times with ethyl acetate. The combined organic layers were dried over MgSO₄, filtered and evaporated. The crude product was purified by column chromatography (silica gel, ethyl acetate, dichloromethane/methanol, methanol).

MS—(M−H⁺) 461.8; (M−H⁻) 459.8

### (1-Aminoethyl)-(2-benzylcarbamoyl-3-phenylpropyl)-phosphinic acid hydrochloride

(2-Benzylcarbamoyl-3-phenylpropyl)-(1-tert-butoxycarbonylaminoethyl)-phosphinic acid (1.06 g, 2.3 mmol) was dissolved in methanolic HCl (1.25 M, 5.5 ml) and stirred at rt over night. The solution was evaporated and the product dried under high vacuum.

MS—(M−H⁺) 361.5

### (2-Benzylcarbamoyl-3-phenylpropyl)-{1 [4-tert-butoxycarbonylamino-5-(2-cyanopyrrolidin-1-yl)-5-oxopentanoylamino]-ethyl}-phosphinic acid

(1-Aminoethyl)-(2-benzylcarbamoyl-3-phenylpropyl)-phosphinic acid hydrochloride (500 mg, 1.26 mmol) and 4-tert-Butoxycarbonylamino-5-(2-cyanopyrrolidin-1-yl)-5-oxopentanoic acid (488 mg, 1.5 mmol) were dissolved in dichloromethane (2.4 ml). Then N-methylmorpholine (0.42 ml, 3.8 mmol), 1-(3-Dimethylaminopropyl)-3-ethylcarbodiimide hydrochloride (315 mg, 1.64 mmol) and hydroxybenzotriazole (203 mg, 1.5 mmol) were added and the reaction mixture stirred at rt over night. The reaction mixture was diluted with ethyl acetate and washed with 1 N HCl and brine. The combined aqueous layers were saturated with NaCl and extracted several times with ethyl acetate. The combined organic layers were dried over MgSO₄, filtered and evaporated. The crude product was purified by column chromatography (silica gel, ethyl acetate, dichloromethane/methanol, methanol).

MS—(M−H⁺) 668.7; (M−H⁻) 666.8

### {1-[4-Amino-5-(2-cyanopyrrolidin-1-yl)-5-oxopentanoylamino]-ethyl}-(2-benzylcarbamoyl-3-phenyl-propyl)-phosphinic acid trifluoroacetate

(2-Benzylcarbamoyl-3-phenylpropyl)-{1 [4-tert-butoxycarbonylamino-5-(2-cyanopyrrolidin-1-yl)-5-oxopentanoylamino]-ethyl}-phosphinic acid (476 mg, 0.7 mmol) was dissolved in trifluoro acetic acid (2 ml) and stirred a rt (reaction control with HPLC-MS). After the reaction was complete, the solution was evaporated and the product dried under high vacuum.

MS—(M−H⁺) 568.8; (M−H⁻) 566.8

### Example 4

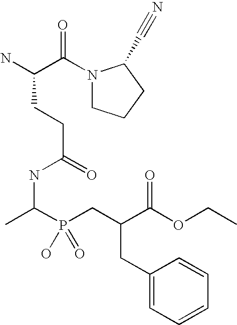

### 3-[(1-Aminoethyl)-hydroxyphosphinoyl]-2-benzylpropionic acid ethylester hydrochloride

2-Benzyl-3-[(1-tert-butoxycarbonylaminoethyl)-hydroxyphosphinoyl]-propionic acid ethyl ester (1 g, 2.5 mmol) was dissolved in methanolic HCl (1.25 M, 6 ml) and stirred at rt over night. The solution was evaporated and the product dried under high vacuum.

MS—(M−H⁺) 300.5; (M−H⁻) 298.5

### 2-Benzyl-3-({1-[4-tert-butoxycarbonylamino-5-(2-cyanopyrrolidin-1-yl)-5-oxopentanoyl]-ethyl}-hydroxyphosphinoyl)-propionic acid ethyl ester

3-[(1-Aminoethyl)-hydroxyphosphinoyl]-2-benzylpropionic acid ethyl ester hydrochloride (0.5 g, 1.5 mmol) and 4-tert-Butoxycarbonylamino-5-(2-cyanopyrrolidin-1-yl)-5-oxopentanoic acid (582 mg, 1.79 mmol) were dissolved in dichloromethane (3 ml). Then N-methylmorpholine (0.5 ml, 4.5 mmol), 1-(3-Dimethylaminopropyl)-3-ethylcarbodiimide hydrochloride (372 mg, 1.94 mmol) and hydroxybenzotriazole (242 mg, 1.79 mmol) were added and the reaction mixture stirred at rt over night. The reaction mixture was diluted with ethyl acetate and washed with 1 N HCl and brine. The combined aqueous layers were saturated with NaCl and extracted several times with ethyl acetate. The combined organic layers were dried over MgSO₄, filtered and evaporated. The crude product was purified by column chromatography (silica gel, ethyl acetate, dichloromethane/methanol, methanol).

MS—(M−H⁺) 607.8; (M−H⁻) 606.0

### 3-({1-[4-Amino-5-(2-cyano-pyrrolidin-1-yl)-5-oxopentanoylamino]-ethyl}-hydroxyphosphinoyl)-2-benzylpropionic acid ethyl ester trifluoroacetate

2-Benzyl-3-({1-[4-tert-butoxycarbonylamino-5-(2-cyanopyrrolidin-1-yl)-5-oxopentanoyl]-ethyl}-hydroxy-phosphinoyl)-propionic acid ethyl ester (497 mg, 0.82 mmol) was dissolved in trifluoroacetic acid (2.3 ml) and stirred a rt (reaction control with HPLC-MS). After the reaction was complete, the solution was evaporated and the product dried under high vacuum.

MS—(M−H⁺) 507.4; (M−H⁻) 505.5

### Example 5

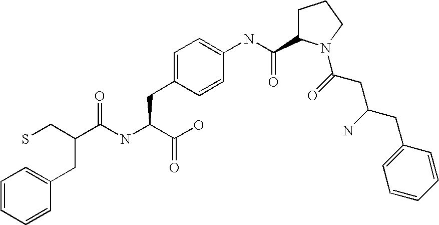

### (1-Hydroxymethyl-2-phenylethyl)-carbamic acid tert.-butyl ester

N-(tert-butoxycarbonyl)-DL-phenylalanine (10 g, 37.7 mmol) was dissolved in THF (38 ml) and cooled to 0° C. Then triethylamine (5.8 ml, 41.5 mmol) and ethyl chloroformate (4 ml, 41.5 mmol) were added and stirred for 1 h at 0° C. The solution was filtered, cooled to 0° C. and a solution of sodium borohydride (1.4 g, 57 mmol) in water (15 ml) was slowly added. After stirring for 30 min at 0° C., the solution was warmed to rt and stirred over night. The reaction mixture was acidified with 1 N HCl and extracted several times with ethyl acetate. The combined organic layers were washed with sat. bicarbonate and brine, dried over MgSO₄, filtered and evaporated.

¹H-NMR (300 MHz, CDCl₃) 7.33-7.21 (m, 5H), 4.9 (bs, 1H), 3.87 (bs, 1H), 3.68-3.52 (m, 2H), 2.87-2.84 (m, 3H), 1.42 (s, 9H)

### Toluene-4-sulfonic acid-2-tert-butoxycarbonylamino-3-phenylpropyl ester

(1-Hydroxymethyl-2-phenyl-ethyl)-carbamic acid-tert-butyl ester (6.9 g, 27.3 mmol) was dissolved in dichloromethane (27 ml) and cooled to 0° C. Then triethylamine (4.2 ml, 30 mmol), dimethylaminopyridine (cat.) and p-toluenesulfonylchloride (6.8 g, 35.5 mmol) were added and the reaction mixture was slowly warmed to rt. After stirring for 2 h at rt, the reaction mixture was acidified with 1 N HCl and extracted several times with dichloromethane. The combined organic layers were dried over MgSO₄, filtered and evaporated. The crude product was used for the next step without further purification.

### (1-Benzyl-2-cyanoethyl)-carbamic acid tert.-butyl ester

Toluene-4-sulfonic acid-2-tert-butoxycarbonylamino-3-phenylpropyl ester (12.4 g, 31 mmol) was dissolved in dimethylformamide (33 ml) and added slowly to a solution of sodium cyanide (4.5 g, 92 mmol) in dimethylformamide (35 ml). The reaction mixture was stirred for 2 d at rt, diluted with water and extracted several times with ethyl acetate. The combined organic layers were dried over MgSO₄, filtered and evaporated. The crude product was purified by column chromatography (silica gel, ethyl acetate/hexanes).

¹H-NMR (300 MHz, CDCl₃) 7.39-7.23 (m, 5H), 4.79 (bs, 1H), 4.12 (bs, 1H), 3.08-3.0 (m, 1H), 2.92-2.84 (m, 1H), 2.9-2.66 (m, 1H), 2.47-2.4 (m, 1H), 1.45 (s, 9H)

### 3-Amino-4-phenylbutyric acid

(1-Benzyl-2-cyanoethyl)-carbamic acid-tert.-butylester (1.55 g, 6 mmol) was dissolved in 6 N HCl (5 ml) and heated under reflux over night. The solution was neutralized with 2 N NaOH and lyophilized. The crude product was used for the next step without further purification.

### 3-tert-Butoxycarbonylamino-4-phenylbutyric acid

3-Amino-4-phenylbutyric acid (1.1 g, 6 mmol) was dissolved in dioxane (12 ml), water (6 ml) and 1 N NaOH (6 ml). Then di-tert-butyl-dicarbonate (1.44 g, 6.6 mmol) was added and the reaction mixture stirred at rt over night. The solution was partially evaporated, acidified with 10% aq. sodium hydrogen sulfate solution, saturated with NaCl and extracted several times with ethyl acetate. The combined organic layers were dried over MgSO₄, filtered and evaporated.

MS—(M−H⁺) 280.3; (M−H⁻) 278.5

### 1-(3-tert-Butoxycarbonylamino-4-phenylbutyryl)-pyrrolidin-2-carbonic acid methyl ester

3-tert-Butoxycarbonylamino-4-phenylbutyric acid (250 mg, 0.9 mmol) and L-proline methyl ester hydrochloride (178 mg, 1.1 mmol) were dissolved in dichloromethane (1 ml). Then N-methylmorpholine (0.3 ml, 2.7 mmol), 1-(3-Dimethylaminopropyl)-3-ethylcarbodiimide hydrochloride (257 mg, 1.34 mmol) and hydroxybenzotriazole (181 mg, 1.34 mmol) were added and the reaction mixture stirred at rt over night. The reaction mixture was concentrated in vacuo and the crude product was purified by column chromatography (silica gel, ethyl acetate/hexanes).

MS—(M−H⁺) 391.9

### 1-(3-tert-Butoxycarbonylamino-4-phenylbutyryl)-pyrrolidin-2-carbonic acid

1-(3-tert-Butoxycarbonylamino-4-phenylbutyryl)-pyrrolidin-2-carbonic acid methyl ester (214 mg, 0.55 mmol) was dissolved in THF (4 ml) and 0.1 M LiOH (5.5 ml) and stirred for 4 h at rt. The solution was partially evaporated, acidified with 10% aq. sodium hydrogen sulfate solution, saturated with NaCl and extracted serveral times with ethyl acetate. The combined organic layers were dried over MgSO₄, filtered and evaporated.

MS—(M−H⁺) 378.0; (M−H⁻) 375.6

### 2-(2-Acetylsulfanylmethyl-3-phenylpropionylamino)-3-(4-aminophenyl)-propionic acid methyl ester

2-Acetylsulfanylmethyl-3-phenylpropionic acid (610 mg, 2.6 mmol) and 4-Amino-L-phenylalanine methyl ester (700 mg, 2.6 mmol) were suspended in dichloromethane (10 ml). Then N-methylmorpholine (0.86 ml, 7.8 mmol), 1-(3-Dimethylaminopropyl)-3-ethylcarbodiimide hydrochloride (590 mg, 3.1 mmol) and hydroxybenzotriazole (460 mg, 3.4 mmol) were added and the reaction mixture stirred at rt over night. The reaction mixture was concentrated in vacuo and the crude product was purified by column chromatography (silica gel, dichloromethane/methanol).

MS—(M−H⁺) 415.6

### 2-(2-Acetylsulfanylmethyl-3-phenylpropionylamino)-3-(4-{[1-(3-tert-butoxycarbonylamino-4-phenylbutyryl)-pyrrolidin-2-carbonyl]-amino}-phenyl)-propionic acid methylester

1-(3-tert-Butoxycarbonylamino-4-phenyl-butyryl)-pyrrolidin-2-carbonic acid (100 mg, 0.27 mmol) and 2-(2-Acetylsulfanylmethyl-3-phenylpropionylamino)-3-(4-aminophenyl)-propionic acid methylester (110 mg, 0.27 mmol) were dissolved in pyridine (1 ml) and cooled to −20° C. Then phosphorus oxychloride (0.03 ml, 0.27 mmol) was added, the reaction mixture stirred for 1 h at −20° C. and then warmed to rt. After 4 h the reaction mixture was concentrated in vacuo and the crude product was purified by column chromatography (silica gel, hexanes/ethyl acetate, dichloromethane/methanol).

### 2-(2-Acetylsulfanylmethyl-3-phenylpropionylamino)-3-(4-{[1-(3-amino-4-phenylbutyryl)-pyrrolidin-2-carbonyl]-amino}-phenyl)-propionic acid methyl ester

2-(2-Acetylsulfanylmethyl-3-phenylpropionylamino)-3-(4-{[1-(3-tert-butoxycarbonylamino-4-phenylbutyryl)-pyrrolidin-2-carbonyl]-amino}-phenyl)-propionic acid methyl ester (73 mg, 0.09 mmol) was dissolved in trifluoro acetic acid (1 ml) and stirred for 1 h at rt. Then the reaction mixture was concentrated in vacuo and the crude product was purified by column chromatography (silica gel, dichloromethane/methanol/ammonium hydroxide).

MS—(M−H⁺) 674.3

### 3-(4-{[1-(3-Amino-4-phenylbutyryl)-pyrrolidin-2-carbonyl]-amino}-phenyl)-2-(2-mercaptomethyl-3-phenylpropionic acid

2-(2-Acetylsulfanylmethyl-3-phenylpropionylamino)-3-(4-{[1-(3-amino-4-phenylbutyryl)-pyrrolidin-2-carbonyl]-amino}-phenyl)-propionic acid methyl ester (53 mg, 0.08 mmol) was dissolved in THF (1.6 ml) and 0.1 N LiOH (2.4 ml) and stirred for 4 h at rt. THF was removed in vacuo and the aqueous solution lyophilized.

MS—(M−H⁺) 618.0; (M−H⁻) 615.5

### Example 6

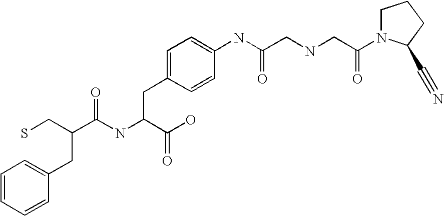

### {tert-Butoxycarbonyl-[2-(2-carbamoylpyrrolidin-1-yl)-2-oxoethyl]-amino}-acetic acid

N-tert-Butoxycarbonyl-iminodiacetic acid (0.5 g, 2.14 mmol) was dissolved in THF (12 ml), dicyclohexylcarbodiimide (442 mg, 2.14 mmol) was added and the reaction mixture stirred at rt over night. Then L-prolinamide (244 mg, 2.14 mmol) was added and the solution warmed to 50° C. for 3 h. The reaction mixture was concentrated in vacuo and the crude product was purified by column chromatography (silica gel, dichloromethane/methanol).

MS—(M−H⁺) 330.5; (M−H⁻) 328.6

### {tert-Butoxycarbonyl-[2-(2-carbamoylpyrrolidin-1-yl)-2-oxoethyl]-amino}-acetic acid methyl ester

{tert-Butoxycarbonyl-[2-(2-carbamoylpyrrolidin-1-yl)-2-oxo-ethyl]-amino}-acetic acid (413 mg, 1.25 mmol) was dissolved in dichloromethane (2 ml), then methanol (0.06 ml, 1.5 mmol), 1-(3-Dimethylaminopropyl)-3-ethylcarbodiimide hydrochloride (288 mg, 1.5 mmol) and dimethylaminopyridine (16 mg, 0.13 mmol) were added and the reaction mixture stirred at rt over night. The reaction mixture was concentrated in vacuo and the crude product was purified by column chromatography (silica gel, hexanes/ethyl acetate, dichloromethane/methanol).

MS—(M−H⁺) 344.5

### {tert-Butoxycarbonyl-[2-(2-cyanopyrrolidin-1-yl)-2-oxoethyl]-amino}-acetic acid methyl ester

{tert-Butoxycarbonyl-[2-(2-carbamoylpyrrolidin-1-yl)-2-oxoethyl]-amino}-acetic acid methyl ester (372 mg, 1.08 mmol) was dissolved in pyridine (9 ml) and cooled to −20° C. Then imidazole (78 mg, 1.14 mmol) and phosphorus oxychloride (0.13 ml, 1.46 mmol) were added and the reaction mixture slowly warmed to rt. After 1 h the reaction mixture was concentrated in vacuo and the crude product was purified by column chromatography (silica gel, hexanes/ethyl acetate, dichloromethane/methanol).

MS—(M−H⁺) 326.6

### {tert-Butoxycarbonyl-[2-(2-cyanopyrrolidin-1-yl)-2-oxoethyl]-amino}-acetic acid

{tert-Butoxycarbonyl-[2-(2-cyanopyrrolidin-1-yl)-2-oxoethyl]-amino}-acetic acid methyl ester (307 mg, 0.94 mmol) was dissolved in THF (7 ml) and 0.1 M LiOH (9.4 ml) and stirred for 4 h at rt. The solution was partially evaporated, acidified with 10% aq. sodium hydrogen sulfate solution, saturated with NaCl and extracted several times with ethyl acetate. The combined organic layers were dried over MgSO₄, filtered and evaporated.

MS—(M−H⁺) 312.5; (M−H⁻) 310.5

### 2-(2-Acetylsulfanylmethyl-3-phenylpropionylamino)-3-[4-(2-{tert-butoxycarbonyl-[2-(2-cyanopyrrolidin-1-yl)-2-oxoethyl]-amino}-acetylamino)-phenyl]-propionic acid methyl ester

2-(2-Acetylsulfanylmethyl-3-phenylpropionylamino)-3-(4-aminophenyl)-propionic acid methyl ester (133 mg, 0.32 mmol) and {tert-Butoxycarbonyl-[2-(2-cyanopyrrolidin-1-yl)-2-oxoethyl]-amino}-acetic acid (100 mg, 0.32 mmol) were dissolved in pyridine (1 ml) and cooled to −20° C. Then phosphorus oxychloride (0.03 ml, 0.32 mmol) was added, the reaction mixture stirred for 1 h at −20° C., warmed to rt and stirred over night. The reaction mixture was poured into icewater and extracted several times with ethyl acetate. The combined organic layers were washed with 10% aq. citric acid solution, sat. bicarbonate and brine, dried over MgSO₄, filtered and evaporated. The crude product was purified by column chromatography (silica gel, hexanes/ethyl acetate, dichloromethane/methanol).

MS—(M−H⁺) 709.2

### 2-(2-Acetylsulfanylmethyl-3-phenylpropionylamino)-3-(4-{2-[2-(2-cyanopyrrolidin-1-yl)-2-oxoethylamino]-acetylamino}-phenyl)-propionic acid methyl ester

2-(2-Acetylsulfanylmethyl-3-phenyl-propionylamino)-3-[4-(2-{tert-butoxycarbonyl-[2-(2-cyanopyrrolidin-1-yl)-2-oxoethyl]-amino}-acetylamino)-phenyl]-propionic acid methyl ester (12 mg, 0.02 mmol) was dissolved in trifluoro acetic acid (1 ml) and stirred for 5 h at rt. The reaction mixture was concentrated in vacuo and the crude product was purified by column chromatography (silica gel, dichloromethane/methanol).

MS—(M−H⁺) 608.8

### 3-(4-{2-[2-(2-Cyanopyrrolidin-1-yl)-2-oxoethylamino]-acetylamino}-phenyl)-2-(2-mercaptomethyl-3-phenylpropionylamino)-propionic acid

2-(2-Acetylsulfanylmethyl-3-phenylpropionylamino)-3-(4-{2-[2-(2-cyanopyrrolidin-1-yl)-2-oxoethylamino]-acetylamino}-phenyl)-propionic acid methyl ester (10 mg, 0.016 mmol) was dissolved in THF (0.4 ml) and 0.1 N LiOH (0.48 ml) and stirred for 4 h at rt. THF was removed in vacuo and the aqueous solution lyophilized.

MS—(M−H⁺) 553.0

### Example 7

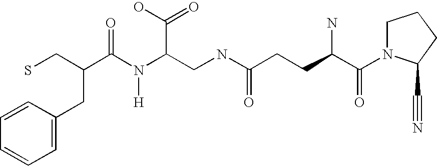

### 2-(2-Acetylsulfanylmethyl-3-phenylpropionylamino)-3-tert-butoxycarbonylamino-propionic acid methyl ester

N-3-(tert-Butoxycarbonylamino)-L-alanine methyl ester (167 mg, 0.76 mmol) and 2-Acetylsulfanylmethyl-3-phenylpropionic acid (181 mg, 0.76 mmol) were dissolved in dichloromethane (2 ml). Then N-methylmorpholine (0.25 ml, 2.3 mmol), 1-(3-Dimethylaminopropyl)-3-ethylcarbodiimide hydrochloride (174 mg, 0.9 mmol) and hydroxybenzotriazole (135 mg, 1 mmol) were added and the reaction mixture stirred at rt over night. The reaction mixture was concentrated in vacuo and the crude product was purified by column chromatography (silica gel, ethyl acetate/hexanes).

MS—(M−H⁺) 439.6

### 2-(2-Acetylsulfanylmethyl-3-phenylpropionylamino)-3-aminopropionic acid methyl ester hydrochloride

2-(2-Acetylsulfanylmethyl-3-phenylpropionylamino)-3-tert-butoxycarbonylamino-propionic acid methylester (96 mg, 0.22 mmol) was dissolved in methanolic HCl (1.25 M, 0.53 ml) and stirred at rt over night. The solution was evaporated and the product dried under high vacuum.

MS—(M−H⁺) 339.5

### 2-(2-Acetylsulfanylmethyl-3-phenylpropionylamino)-3-[4-tert-butoxycarbonylamino-5-(2-cyanopyrrolidin-1-yl)-5-oxopentanoylamino]-propionic acid methyl ester

2-(2-Acetylsulfanylmethyl-3-phenylpropionylamino)-3-aminopropionic acid methylester hydrochloride (75 mg, 0.2 mmol) and 4-tert-Butoxycarbonylamino-5-(2-cyanopyrrolidin-1-yl)-5-oxopentanoic acid (80 mg, 0.2 mmol) were dissolved in dichloromethane (1 ml). Then N-methylmorpholine (0.07 ml, 0.6 mmol), 1-(3-Dimethylaminopropyl)-3-ethylcarbodiimide hydrochloride (46 mg, 0.24 mmol) and hydroxybenzotriazole (35 mg, 0.26 mmol) were added and the reaction mixture stirred at rt over night. The reaction mixture was concentrated in vacuo and the crude product was purified by column chromatography (silica gel, ethyl acetate/hexanes, ethyl acetate).

MS—(M−H⁺) 647.3

### 2-(2-Acetylsulfanylmethyl-3-phenylpropionylamino)-3-[4-amino-5-(2-cyanopyrrolidin-1-yl)-5-oxopentanoylamino]-propionic acid methyl ester

2-(2-Acetylsulfanylmethyl-3-phenylpropionylamino)-3-[4-tert-butoxycarbonylamino-5-(2-cyanopyrrolidin-1-yl)-5-oxopentanoylamino]-propionic acid methyl ester (32 mg, 0.05 mmol) was dissolved in trifluoro acetic acid (0.63 ml) and water (0.03 ml) and stirred for 4 h at rt. The reaction mixture was concentrated in vacuo and the crude product was purified by column chromatography (silica gel, dichloromethane/methanol/ammonium hydroxide).

MS—(M−H⁺) 546.7

### 3-[4-Amino-5-(2-cyanopyrrolidin-1-yl)-5-oxopentanoylamino]-2-(2-mercaptomethyl-3-phenylpropionylamino)-propionic acid

2-(2-Acetylsulfanylmethyl-3-phenylpropionylamino)-3-[4-amino-5-(2-cyanopyrrolidin-1-yl)-5-oxopentanoylamino]-propionic acid methyl ester was dissolved in THF (0.28 ml) and 0.1 N LiOH (1 ml) and stirred for 7 h at rt. THF was removed in vacuo and the aqueous solution lyophilized.

MS—(M−H⁺) 491.6; (M−H⁻) 490.9

### Example 8

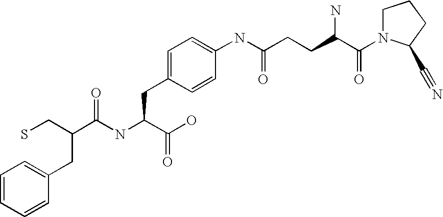

### 2-(2-Acetylsulfanylmethyl-3-phenylpropionylamino)-3-{4-[4-tert-butoxycarbonylamino-5-(2-cyanopyrrolidin-1-yl)-5-oxopentanoylamino]-phenyl}-propionic acid methyl ester

2-(2-Acetylsulfanylmethyl-3-phenylpropionylamino)-3-(4-aminophenyl)-propionic acid methyl ester (127 mg, 0.31 mmol) and 4-tert-Butoxycarbonylamino-5-(2-cyanopyrrolidin-1-yl)-5-oxopentanoic acid (100 mg, 0.31 mmol) were dissolved in pyridine (1 ml) and cooled to −20° C. Then phosphorus oxychlorid (0.03 ml, 0.34 mmol) was added, the reaction mixture stirred for 2 h at −20° C., warmed to rt and stirred over night. The reaction mixture was poured into ice water and extracted several times with ethyl acetate. The combined organic layers were washed with 10% aq. citric acid solution, sat. bicarbonate and brine, dried over MgSO₄, filtered and evaporated. The crude product was purified by column chromatography (silica gel, hexanes/ethyl acetate).

MS—(M−H⁺) 722.5; (M−H⁻) 720.8

### 2-(2-Acetylsulfanylmethyl-3-phenylpropionylamino)-3-{4-[4-amino-5-(2-cyanopyrrolidin-1-yl)-5-oxopentanoylamino]-phenyl}-propionic acid methyl ester

2-(2-Acetylsulfanylmethyl-3-phenylpropionylamino)-3-{4-[4-tert-butoxycarbonylamino-5-(2-cyanopyrrolidin-1-yl)-5-oxopentanoylamino]-phenyl}-propionic acid methylester (52 mg, 0.31 mmol) was dissolved in trifluoro acetic acid (1 ml) and water (0.05 ml) and stirred for 4 h at rt. The reaction mixture was concentrated in vacuo and the crude product was purified by column chromatography (silica gel, dichloromethane/methanol).

MS—(M−H⁺) 623.0

### 3-{4-[4-Amino-5-(2-cyanopyrrolidin-1-yl)-5-oxopentanoylamino]-phenyl}-2-(2-mercaptomethyl-3-phenylpropionylamino)-propionic acid

2-(2-Acetylsulfanylmethyl-3-phenylpropionylamino)-3-{4-[4-amino-5-(2-cyanopyrrolidin-1-yl)-5-oxopentanoylamino]-phenyl}-propionic acid methyl ester (8 mg, 0.013 mmol) was dissolved in THF (0.2 ml) and 0.1 N LiOH (0.4 ml) and stirred for 6 h at rt. THF was removed in vacuo and the aqueous solution lyophilized.

MS—(M−H⁺) 567.2; (M−H⁻) 564.8

All publications cited in the specification, both patent publications and non-patent publications, are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. Novel compounds with the general formula A-L-B (I), A-L-C (II), A-L-D (III) where
A is an inhibitor of dipeptidyl peptidase IV or a pharmacophore thereof;
B is an inhibitor of neprilysin or a pharmacophore thereof;
C is an inhibitor of angiotensin converting enzyme or a pharmacophore thereof;
D is an inhibitor of vasopeptidases (especially NEP and ACE) or a pharmacophore thereof; and
L is a linker, or a pharmaceutically acceptable salt, solvent, or formulation.

2. A compound that has one or more functional groups that are easily changed or removed under physiological conditions during or after administration to a mammalian patient to yield a compound having the general formula A-L-B (I), A-L-C (II), A-L-D (III) as defined in claim 1 or pharmaceutically acceptable salts thereof.

3. A pharmaceutical composition comprising a compound of claim 1 and a pharmaceutically acceptable carrier.

4. A method for treating, controlling or preventing non-insulin dependent (Type 2) diabetes mellitus in a mammalian patient in need of such treatment which comprises administering to said patient a therapeutically effective amount of a compound of claim 1.

5. A method for treating, controlling or preventing hyperglycemia in a mammalian patient in need of such treatment which comprises administering to said patient a therapeutically effective amount of a compound of claim 1.

6. A method for treating, controlling or preventing insulin resistance in a mammalian patient in need of such treatment which comprises administering to said patient a therapeutically effective amount of a compound of claim 1.

7. A method for treating, controlling or preventing Syndrome X in a mammalian patient in need of such treatment which comprises administering to said patient a therapeutically effective amount of a compound of claim 1.

8. A method for treating, controlling or preventing one or more conditions selected from the group consisting of: (1) hyperglycemia, (2) low glucose tolerance, (3) insulin resistance, (4) obesity, (5) lipid disorders, (6) dyslipidemia, (7) hyperlipidemia, (8) hypertriglyceridemia, (10) low HDL levels, (11) high HDL levels, (12) atherosclerosis and its sequelae, (13) vascular restenosis, (14) irritable bowel syndrome, (15) inflammatory bowel disease, including Crohn's disease and ulcerative colitis, (16) other inflammatory conditions, (17) pancreatitis, (18) abdominal obesity, (19) neurodegenerative disease, (20) retinopathy, (21) nephropathy, (22) neuropathy, (23) syndrome X, (24) ovarian hyperandrogenism (polycystic ovarian syndrome), (25) type 2 diabetes, (26) growth hormone deficiency, (27) neutropenia, (28) neuronal disorders, (29) tumor metastasis, (30) benign prostatic hypertrophy, (31) gingivitis, (32) hypertension, (33) osteoperosis, and other disorders where insulin resistance is a component or that may be treated by inhibitors of DPP-IV, NEP, and/or ACE.

9. The use of a compound of the general formula A-L-B (I), A-L-C (II), A-L-D (III) as defined in claim 1 or a pharmaceutically acceptable salt thereof for the manufacture of a medicament for the treatment of type 2 diabetes.
